(19) United States
(12) Patent Application Publication
(10) Pub. No.: US 2011/0171636 A1
(43) Pub. Date: Jul. 14, 2011

(54) MONO- AND MULTI-ELEMENT CODED LIBS ASSAYS AND METHODS

(75) Inventors: Noureddine Melikechi, Dover, DE (US); Yuri Markushin, Dover, DE (US)

(73) Assignee: DELAWARE STATE UNIVERSITY, Dover, DE (US)

(21) Appl. No.: 13/051,504

(22) Filed: Mar. 18, 2011

Related U.S. Application Data
(63) Continuation-in-part of application No. PCT/US2009/056798, filed on Sep. 14, 2009.
(60) Provisional application No. 61/098,376, filed on Sep. 19, 2008.

(51) Int. Cl. G01N 33/53; G01J 3/30; C12Q 1/68; B82Y 15/00

(52) U.S. Cl. 435/6.1; 356/318; 436/164; 435/7.1; 977/773; 977/700; 977/920

(57) ABSTRACT

Methods for tagging an object with an element-coded particle and identifying the object based on the element code are described. LIBS analysis can be used with the methods to provide a high resolution system for identifying and quantifying objects with great specificity. Objects can include biological and chemical molecules.

### CROSS REFERENCE TO RELATED APPLICATIONS

This application is a continuation-in-part of International Application PCT/US2009/056798, filed Sep. 14, 2009, which claims priority to U.S. Provisional Application No. 61/098,376, filed on Sep. 19, 2008, the contents of each of which are incorporated herein in their entireties for all purposes.

### STATEMENT REGARDING FEDERALLY SPONSORED RESEARCH

This invention was made with Government support under CDMRP award no. OC050108 from the Department of Defense and grant no. 0630388 from the National Science Foundation. The Government has certain rights in this invention.

### BACKGROUND OF THE INVENTION

Laser-induced breakdown spectroscopy (LIBS) is a valuable tool for identifying components of a sample. High sensitivity, simplicity and specificity make LIBS a powerful technique for the detection of chemical agents or pollutants from the level of ppm to ppb. (K. Song, Y. Lee and J. Sneddon, “Applications of laser-induced breakdown spectrometry”, Appl. Spectros. Rev. 32, 183-235 (1997); G. Arca, A. Ciucci, V. Palleschi, S. Rastelli and E. Tognini, “Trace element analysis in water by laser-induced breakdown pectroscopy”, Appl. Spectros. 51, 1102-1105 (1997)). Compared with the numerous elemental analytical techniques available, LIBS provides many advantages. LIBS requires much smaller sample volumes and minimal sample preparation. LIBS provides real-time spectra, does not require the use of time-of-flight devices and is easy to implement. In addition, elements analyzed by LIBS have extremely narrow emission bandwidths and characterization of each chemical element, as defined by a unique series of emission lines, is highly specific. As a result, LIBS is one of the most effective techniques for multi-element analysis of samples. LIBS has accordingly attracted significant attention in fields such as environmental analysis, forensics, and, more recently, in biological warfare. (A. Kumar, F. Y. Yueh, J. P. Singh, and S. Burgess, “Characterization of malignant tissue cells by laser-induced breakdown spectroscopy”, Appl. Opt. 43, 5399-5403 (2004); A. C. Samuels, F. C. DeLucia Jr., K. L. McNesby, and A. W. Miziolek, “Laser-induced breakdown spectroscopy of bacterial spores, molds, pollens, and protein: initial studies of discrimination potential”, Appl. Opt. 42, 6205-6209 (2003); A. R. Boyain-Goitia, D. C. S. Beddows, B. C. Griffiths, and H. H. Telle, “Single-pollen analysis by laser-induced breakdown spectroscopy and Raman microscopy”, Appl. Opt. 42, 6119-6132 (2003); S. Morel, N. Leone, P. Adam, and J. Amouroux, “Detection of bacteria by time-resolved laser-induced breakdown spectroscopy”, Appl. Opt. 42, 6184-6191, (2003); M. B. Gretzer, A. W Partin, D. W. Chan, and R. W Veltri, “Modern tumor marker discovery in urology: Surface Enhanced Laser Desorption and Ionization (SELDI)”, Rev. Urol. 5, 81-89 (2003); J. Hybl, G. Lithgow and S. Buckley, “Laser-induced break-down spectroscopy detection and classification of biological aerosols”, Appl. Spectros 57, 1207-1215 (2003)).

LIBS consists of focusing a laser pulse on the sample of interest using a power density greater than the breakdown threshold of the sample to create a plasma at temperatures of around 10,000-20,000° K. This results in chemical breakdown of the sample components into their atomic constituents. As the plasma cools, it undergoes atomic and ionic emissions that are spectrally resolved to yield information on the elemental composition of the samples.

Quantum dot (QD) nanocrystals are fluorescent labels that can be excited with UV or violet light, as well as with longer-wavelength light, and exhibit long Stokes shifts and relatively narrow emission peaks. QDs have been encapsulated in amphiphilic polymers and bound to tumor-targeting ligands and drug delivery vesicles for targeting, imaging and treating tumor cells. QDs have been covalently linked to various biomolecules such as antibodies, peptides, nucleic acids and other ligands for fluorescence probing applications, for example, Invitrogen offers primary antibody-quantum dot conjugates and secondary detection reagents. (Sandeep Kumar Vashist, Rupinder Tewari and Roberto Raiteri, “Review of Quantum Dot Technologies for Cancer Detection and Treatment”, The AZo Journal of Nanotechnology Online, Volume 2, September 2006, pp. 1-14, azonano.com/Details.asp?ArticleID=1726; “Expand your horizons in flow cytometry with Qdot nanocrystals,” Invitrogen Corporation brochure, tools.invitrogen.com/content/sfs/brochures/F074015Qdot_primaries_pp.pdf).

While both quantum dots and LIBS can be used to analyze components in a sample, the LIBS technique has greater resolution because atomic emission spectra of plasma are much narrower than fluorophore emissions. A typical spectral line width for LIBS applications ranges from about 0.1-10 nm (J. E. Carranza, K. Iida, D. W. Hahn, “Conditional data processing for single-shot spectral analysis by use of laser-induced breakdown spectroscopy”, Appl. Opt. 42, 6022-6028, (2003)), whereas a typical spectral line width for quantum dots ranges from about 20-40 nm (T. M. Jovin, “Quantum dots finally come of age”, Nature Biotechnology 21, 32-33, (2003)).

### SUMMARY OF THE INVENTION

Methods for identifying and/or tagging an object are described. These include (1) a method of identifying a biomarker in a biological sample comprising the steps of a) reacting a biological sample containing a biomarker with a plurality of element-coded particles each comprising a compound that binds to the biomarker, b) removing unbound element-coded particles from the sample, and c) detecting the element-coded particles in the sample using an optical system; (2) a method of identifying multiple biomarkers simultaneously in a biological sample comprising the steps of a) reacting a biological sample containing more than one biomarker with a plurality of element-coded particle types, wherein each particle type comprises a specific element code and a compound that binds to a discrete biomarker, c) removing unbound element-coded particles from the sample, and d) detecting the element-coded particles in the sample using an optical system; and (3) a method of tagging an object comprising the step of a) attaching one or more element-coded particle as a tag to the object to produce a tagged object, b) analyzing the tagged object by laser-induced breakdown spectroscopy (LIBS) to produce an emission spectrum from the tag, and c) identifying the object by correlating the tagged object with the emission spectrum of the tag. Each method may further comprise quantifying the element coded particles in the sample.

### BRIEF DESCRIPTION OF THE DRAWINGS

FIG. 1 is a graph showing the LIBS analysis of protein A-coated Si particles (solid line) and the silicon standard emission spectrum from the LIBS library (dotted line).

FIG. 2 is a graph showing the LIBS analysis of (a) immunoconjugated Si particles bound to agarose affinity resin particles bearing CA 125; (b) agarose affinity resin particles bearing CA 125; and (c) immunoconjugated Si particles pre-incubated with CA 125 before binding agarose affinity resin particles bearing CA 125.

FIG. 3 is a schematic of the multi-element coded LIBS assay protocol for analysis of a biomarker in a sample containing the biomarker (positive test).

FIG. 4 is a schematic of the multi-element coded LIBS assay protocol for analysis of a biomarker in a sample that does not contain the biomarker (negative test).

FIG. 5 is a graph of a LIBS detection of biotin-coated, Fe microparticles vs. avidin concentration

FIG. 6 is a graph of a LIBS detection of two-element (Si, Fe) coded microparticles. Dashed line—Fe and Si containing particles; dotted line particles containing only Si; dot-dash line—particles containing only Fe; solid line—empty filter.

FIG. 7 is a graph of a LIBS detection of two-element (Si, Fe) coded microparticles. Dashed line—Fe and Si containing particles; dotted line particles containing only Si; dot-dash line—particles containing only Fe; solid line—empty filter.

FIG. 8 is a graph of a LIBS assay for Si-coded leptin determination.

FIG. 9 is a graph of a LIBS assay for Fe-coded CA125 determination.

FIG. 10 shows the fragment of LIBS spectra around 288.1 nm silicon emission line in the two-element (Si and Fe) Tag-LIBS assay for detection of CA 125. The solid line represents LIBS of the empty filter. Other lines represent various concentrations of CA 125 in a solution: dash line—control sample with no CA 125, long dash line—10 U/ml, dot-dash line—50 U/ml, dot line—250 U/ml, dot-dot-dash line—1000 U/ml.

FIG. 11 shows the fragment of background subtracted LIBS spectra around 280.2 nm Gold emission line in the two-element (Au and Fe) Tag-LIBS assay for detection of avidin in human blood plasma. Concentration of avidin: 0-0 ppb (control sample), 1-6.4 ppb, 2-64.5 ppb, 3-322.4 ppb, 4-644.8 ppb, 5-1483.1 ppb, 6-2321.4 ppb, 7-3224.2 ppb, 8-6448.4 ppb.

### DETAILED DESCRIPTION OF THE INVENTION

LIBS analysis may be utilized to identify element-tagged markers and to create a spectral “barcode” of elements used to tag specific markers. The high resolution of the LIBS system provides an improved method to detect, identify and quantify multiple elements in a single sample. Embodiments of the invention described below comprise methods of using particles containing one or more elements that can be assayed via LIBS analysis to specifically tag markers for subsequent identification and, optionally, quantification.

The particles can comprise one or more chemical elements to produce an element code. These are referred to herein as “element-coded particles”. For examples of elements, a table of chemical elements and their LIBS spectra can be found in solartii.com/analytical_instruments/lea-s500, which is incorporated herein by reference. In addition, the National Institute of Standards and Technology Physics Laboratory has published a handbook to provide atomic spectroscopic data, which is available at physics.nist.gov/physrefdata/handbook/; and the Center for Research and Education in Optical Sciences and Applications has established a database for LIBS spectra at creosa.desu.edu.libs.html, both of which are incorporated herein by reference in their entirety for all purposes.

The element-coded particles can have any shape, e.g., strings, rods, tubes, threads, spheres, rings, plates, bricks, strips, etc. The element-coded particles are nanometer, micrometer, or millimeter sized particles, ranging from 10 nm to 10 mm, preferably from 10 nm to 1 mm. Element-coded particles in this size range are commercially available or can be prepared by known methods. Commercial sources include Nanocs Inc., New York, N.Y.; Spherotech, Inc., Lake Forest, Ill.; and Thermo Fisher Scientific Inc., Rockford, Ill. Methods for making the particles can be found, for example, in WO/2006/135384 and U.S. Pat. Nos. 5,149,496; 5,545,360; 5,628,945; 6,232,372; 7,341,757; 7,367,999; 7,368,130; and 7,381,467, which are incorporated herein by reference.

Element-coded particles can be porous, solid, flexible, amorphous, multi-layered, etc. as appropriate for a specific use. Composite element-coded particles may be made by connecting particles together via chemical or electrostatic bonds, magnetic forces, encapsulation, or by physical bonds such as glue, alloys, co-melting, wrapping, pressing, mechanically connecting, etc., according to known methods. Single particles comprising different element codes may also be used together in a mixture. Once constructed, particles can be suspended and stored in a liquid, solid, or gas medium or in a vacuum.

The element code for an element-coded particle is created by the elements present in the particle. A particle can contain one or more elements, or nanoparticles and microparticles bearing one or more elements can be combined into larger, composite particle structures to produce highly specific spectroscopic bar codes. Ideally, composite element-coded particles having highly specific spectroscopic bar codes comprise combinations of elements in a predetermined quantity and ratio that is unique and not naturally occurring in the source to be tagged with the composite particle. Similarly, even when mono element-coded particles comprising only a single element are used, an element is selected that is not naturally occurring in the source. Thus, when LIBS analysis is performed and produces the signature spectroscopic bar code corresponding to the unique combination of elements in the composite particle or corresponding to the single element of a mono element-coded particle, there can be no question of the presence of the composite particle structure or mono element-coded particle, respectively. For example, there are about 80 known metals. Combining 11 different elements (i.e. iron, gold, silver, platinum, aluminum, titanium, vanadium, nickel, zinc, tin and copper) gives more then 1000 types of composite particles. Some of them are well known alloys such as brass (copper and zinc), bronze (copper and tin), and duralumin (aluminum and copper). Each of these composites is unique in chemical content and may be used as a micro-tag for labeling and detecting molecules of interest in the multi-element coded LIBS assay.

The sensitivity of the multi-element coded LIBS assay can be optimized by increasing the size of the element-coded particles to amplify the signal, increasing the number of element-coded particles in the assay, and selecting elements with brighter emission lines. A fully optimized assay would be capable of detecting a single protein molecule.

The element-coded particles can be modified or derivatized for attachment to objects of interest, including, but not limited to, biological molecules, cells, tissues organisms, other chemical molecules, particles, surfaces, fabric, paper, and membranes. Biological molecules include peptides, proteins, amino acids, nucleic acids, nitrogenous bases, hydrocarbons, polysaccharides, fatty acids, lipids and polymers of molecular subunits. In general, the element-coded particles are surface modified with organic layers to reduce hydrophobicity and to provide reactive groups for subsequent conjugation to the object to be labeled by the element-coded particle. Methods for surface modification are known in the art, e.g., U.S. Pat. No. 4,715,986.

For example, in one embodiment the object to be labeled is a biomolecule, such as a protein. The element-coded particles can be surface modified to contain reactive groups such as amines, aldehyde, carboxyl and thiol groups, polyethylene glycol (PEG), or short peptides. The surface-modified element-coded particles can then be chemically conjugated or coated with biologically interactive molecules such as streptavidin, biotin, protein A, protein G, protein L, IgG molecules, specific antibodies, receptor molecules, specific peptides, specific oligonucleotides, etc. Methods for conjugation and coating are known in the art, e.g., piercenet.com/files/1601361Crosslink.pdf; piercenet.com/files/2066 as4.pdf); Vaibhav S. Khire, Tai Yeon Lee, and Christopher N. Bowman, Surface Modification Using Thiol-Acrylate Conjugate Addition Reactions, Macromolecules, 40 (16), 5669-5677, 2007.

Cross-linking and spacer molecules may be used to properly orient the interactive molecule and to avoid steric hindrance. Surface-modified element-coded particles and services for modifying an element-coded particle surface are also commercially available, e.g., Nanocs Inc., New York, N.Y.; Spherotech, Inc., Lake Forest, Ill.; Thermo Fisher Scientific Inc., Rockford, Ill.; Bangs Laboratories Inc., Fishers, Ind.; Chemicell GmbH, Berlin, Germany.

In one embodiment, the element-coded LIBS assay provides an improved system for detecting and quantifying biomarkers in biological samples. The improved resolution and sensitivity of the assay compared with existing detection methods will enable earlier detection of disease biomarkers, such as cancer biomarkers. The type of biomarker is not limited and can be any biological marker for which a specific binding partner can be provided. Specific binding pairs include, but are not limited to, ligands and antibodies or antibody fragments, proteins and receptors, nonprotein hormones and receptors, biotin and avidin derivatized molecules, IgG and Proteins A, G, and L, DNA and DNA-binding proteins, complementary oligonucleotides. Specific binding partners can also include natural or synthetic small molecules, peptides, oligonucleotides, proteins, polysaccharides, and lipids. An example of this embodiment is described in Markushin, et al., “LIBS-based multi-element coded assay for ovarian cancer application,” Proc. of SPIE 7190: 719015-1-79015-6, 2009.

In this embodiment, a sample of biological tissue or fluid believed to contain a specific biomarker is incubated with an element-coded particle or mixture of element-coded particles bearing interactive molecules that are able to bind with the biomarker. Unbound element-coded particles are washed away and the bound element-coded particles are assayed and quantified using LIBS, as described in Example 4.

The biological sample can be any body fluid, such as blood, urine, saliva, amniotic fluid, etc., or can be a cell, tissue, organism, tissue homogenate, growth medium, or other solution containing biomolecules. Tissue and organisms can be sectioned, homogenized, or intact. Tissues are incubated with the element-coded particles in an appropriate buffer. Biological fluids can be used directly or can be buffered for incubation with the element-coded particles. The incubation mixture can contain a blocking agent, such as bovine serum albumin, to prevent nonspecific binding of the element-coded particles. Reaction times are determined empirically, but can be estimated based on the known affinity of a specific binding molecule for a specific biomarker, the volume of the incubation mixture, and the selected temperature of the incubation. For a small volume incubation comprising binding partners with high affinity and nanometer sized particles, very short incubations are sufficient, i.e., milliseconds. The incubation mixture can be stirred or shaken or allowed to stand. Incubations may be performed on slides, in culture dishes, in microwell plates, in tubes, in tubing, or with any appropriate container or substratum.

Unbound or bound element-coded particles or other components of the reaction (e.g., salts, cell debris) are removed by any appropriate means, such as filtration, centrifugation, spin-filtration, affinity or exclusion chromatography, washing, or by applying other types of forces, such as electric and magnetic fields. Electromagnets and permanent magnets (e.g., neodymium NdFeB magnets, K&J Magnetics, Inc., Jamison, Pa.), filter plates, such as the MultiScreen Ultracel-10 filter plate (Millipore Corp., Billerica, Mass.) can be used for high throughput sample preparation. Bound aggregates of element-coded particles and molecules of interest may also be removed prior to the following analysis.

After removal of unbound element-coded particles and, optionally, other components, the sample is analyzed by LIBS using standard techniques. Basically, the sample is placed in a sample chamber of a LIBS system. Liquid samples can be adsorbed onto a filter surface for the analysis. A laser is focused onto the sample and pulsed to generate a plasma and dissociate the sample into atomic species. One or more atomic emission spectra are produced based on the types of element-coded particles in the sample. Commercially available software programs are used to identify and quantify the types of element-coded particles present in the sample. The spectral “bar codes” are then compared with the types of element-coded particles mixed with the sample and “translated” to determine which biomarkers are present in the sample. The specificity of the LIBS assay can be tested by comparison with a competition assay, wherein the sample is preincubated with a specific-binding partner prior to addition of the element-coded particles, as described in Example 2.

Commercially available laser induced breakdown spectrometers include the LEAS500 from Solar TII; LIBScan 50/100 and Portable LIBS System Model 0117 from Applied Photonics Ltd., Ocean Optics LIBS-ELITE, and the PORTA-LIBS-2000 System from StellarNet Inc. Commercially available systems can be optimized for particular applications and laser and detection components can also be combined with newly developed systems for sample handling, and analysis and diagnostics, such as biochemical analyzers, biochip readers, etc. The LIBS system can be fully automated. Portable systems are available for field applications.

Although the LIBS system provides the greatest resolution and sensitivity, other optical measuring techniques can be used to detect and quantify the element-coded particles, e.g., Atomic-Absorption-Spectrometry (AAS), Flame-AAS (FAAS), Graphite-Furnace-AAS (GFAAS), Cold-Vapour-AAS (CVAAS), Hydride-AAS (HyAAS), Atomic-Emission-Spectrometry with Inductively Coupled Plasma (ICP-OES), Mass-Spectrometry with Inductively Coupled Plasma (ICP-MS); X-Ray Fluorescence Spectroscopy, Scanning Electron Microscopy-Energy Dispersive X-Ray Fluorescence Spectroscopy (SEM-EDX).

The invention is not limited to detection and quantification of biomarkers in biological samples. The multi-element coded LIBS assay can also be used to tag or label any object of interest, such as sensors, chips, activated surfaces, fabric, paper, membranes, chemical compounds, etc. For example, element-coded particles can be used in methods such as immuno-blotting, chromatography, or electrophoreses for labeling analytes of interest. As described above, the element-coded particles are modified for attachment to the object of interest and are later used to identify the object.

### EXAMPLES

### 1. Preparation of Immunoconjugated Si Particles

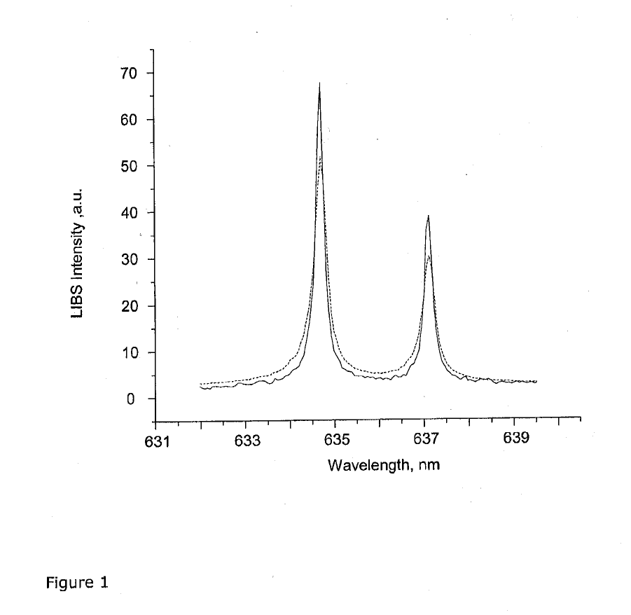

Commercially available 1.5 μm diameter protein A-coated Si particles (G. Kisker GbR, Steinfurt, Germany) were collected from aqueous buffer using centrifugal filters with a molecular weight cut-off of about 100 kD (Steriltech Corp., Kent, Wash.). The particles were adsorbed onto a filter surface and analyzed with LIBS. Results were compared with the silicon standard emission spectrum from the LIBS library (Rock, et al., “Elemental analysis of laser induced breakdown spectroscopy aided by an empirical spectral database” Applied Optics. 47: G99-G104, (2008); creosa.desu.edu/LIBS.html) as shown in FIG. 1. The protein A-coated Si particles (dotted line) elicited a spectrum identical to the standard emission spectrum for silicon (solid line).

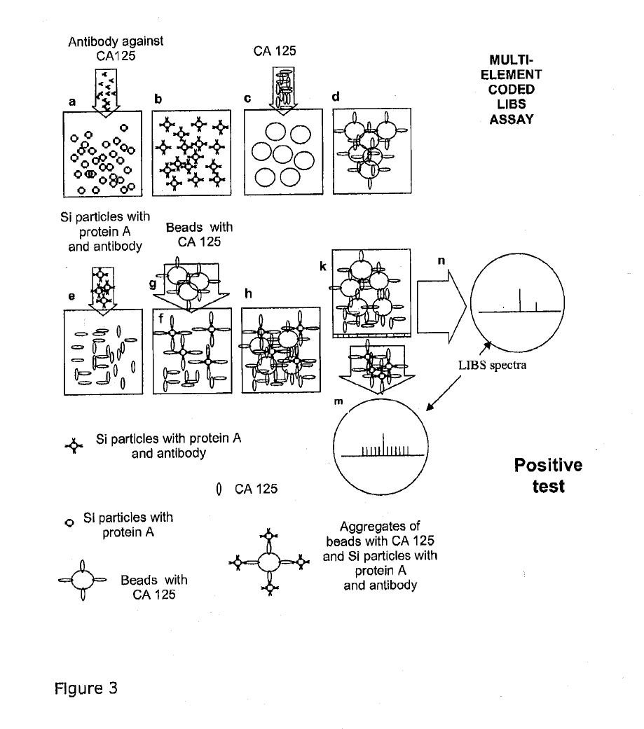

IgG antibodies specific for the ovarian cancer antigen, CA 125, (Biodesign Internat'I., Saco, Me.) were allowed to bind to the protein A-coated Si particles (G. Kisker GbR, Germany). Protein A-coated Si micro-particles were incubated with antibody to CA 125 to allow the antibody to attach to the protein A, and unbound antibody was removed by filtering the incubation mixture through a 0.45 μm filter (FIGS. 3 a,b and 4 a,b).

### 2. Preparation of Agarose Beads Bearing CA 125 Antigen

CA 125 protein (Biodesign Internat'l, Saco, Me.) was covalently attached to cross-linked 4% beaded agarose (20-100 μm diameter), pre-activated with aldehyde groups (AminoLink Coupling Resin, Pierce), via the formation of stable bonds between the aldehyde groups of the agarose and amine groups of the protein. Unbound CA 125 was removed by filtration through a 5 μm filter (Ultrafree-MC SV 5 μm centrifuge filter, Millipore Corp., Billerica, Mass.). (FIGS. 3 c,d and 4 c,d).

### 3. LIBS Analysis of CA 125 Bound to Immunoconjugated Si Particles

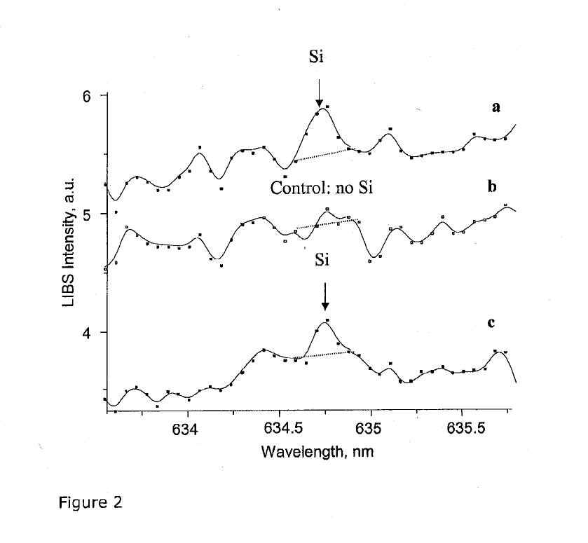

Si particles immunoconjugated to antibody for CA 125 were allowed to bind with agarose beads bearing CA 125 protein. After the incubation, unbound Si particles were removed by size filtration. Sample containing Si particles bound to CA 125 on agarose beads was then analyzed by LIBS. Results are shown in FIG. 2a.

CA 125 was bound to agarose beads as described in Example 2. The CA 125 bound beads were analyzed by LIBS. Results are shown in FIG. 2b.

The LIBS immunoassay was tested in a competition protocol. Si particles immunoconjugated to antibody for CA 125 were pre-incubated with a solution containing free CA 125 and allowed to bind the CA 125. Unbound CA 125 was then removed from the solution by size filtration. The pre-incubated Si particles were then incubated with agarose beads carrying CA 125. Si particles bound with agarose beads were separated from particles not bound to agarose beads by size filtration. The sample containing Si particles bound to agarose beads was then analyzed by LIBS. Results are shown in FIG. 2c.

LIBS spectra were obtained by focusing the light beam generated from a 10 ns ND-YAG infrared pulse laser operating at 1064 nm on the sample. Light pulses ablate the sample creating short-lived plasma. Light emitted by the plasma during cooling is collected by a bundle of optical fibers and delivered to an OOI spectrometer (190-970 nm) for analysis.

FIG. 2 demonstrates that the LIBS immunoassay is capable of specifically recognizing and quantifying a biomarker, such as CA 125, that is bound to a particle containing a detectable element such as Si. The area under the Si spectral peak (at 634.75 nm) is proportional to the amount of biomarker bound to the Si particles as shown by comparing spectrum “a” with spectrum “c”. Pre-incubation competition reduced the amount of CA 125 bearing agarose beads bound to the Si particles. The area under the Si peak in spectrum “c” is reduced accordingly. When no Si is present in the sample, no Si peak greater than the background level is observed (spectrum “b”). Although the amplitudes of 634.75 nm peaks of spectra “a” and “b” are relatively small (signal-to-noise ratio is about 2), the areas under the peak of spectrum “a” (about 1400 a.u.) and spectrum “c” (about 700 a.u.) are greater than in the control, CA 125-bearing agarose beads only, of spectrum “b” (about 0 a.u.).

### 4. Bead Based LIBS Immunoassay for Detection of a Single Biomarker, CA 125

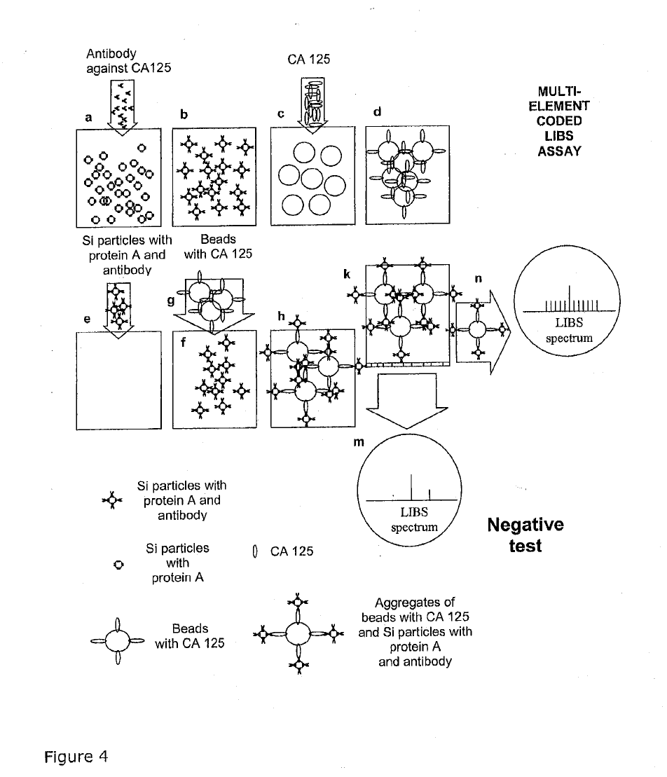

Antibody-bound Si microparticles are incubated with an aqueous sample containing CA 125 (FIG. 3e, positive test) or with an aqueous sample lacking CA 125 (FIG. 4e, negative test). During incubation, CA 125 in the sample will bind to the antibody on the Si microparticles (FIGS. 3f and 4f). Agarose beads with attached CA 125 are then added to the incubation mixture (FIGS. 3g and 4g) to allow unbound Si microparticles to bind to the CA 125 on the agarose beads (FIGS. 3h and 4h). Si particles and Si particle-bound agarose beads are then separated by size filtration (FIGS. 3k and 4k). Si particles bound to agarose beads (residue particles) are analyzed by LIBS (FIGS. 3n and 4n) and Si particles not bound to agarose beads (filtrate particles) are also analyzed by LIBS (FIGS. 3m and 4m).

The quantity of CA-125-bound Si microparticles (filtrate particles) will be directly related to the concentration of the CA 125 biomarker in the sample, and the quantity of Si microparticles bound to agarose beads (residue particles) will be inversely proportional to the concentration of CA 125 in the sample.

### 5. Two-Element-Coded Composite Micro-Particles

Test tubes (0.5 mL) equipped with 5 μm pore filters (Millipore) were used to separate single and aggregated particles. In the experiments with particle assays every step of incubation was followed by a washing step to remove unbound reactants and then a centrifugation step to separate single and aggregated particles. Single and aggregated particles were separated from each other into separate fractions to be analyzed by LIBS.

A LIBS spectral database was employed to identify chemical elements in a pattern of the experimental emission spectra (S. Rock, A. Marcano, Y. Markushin, C. Sabanayagam, N. Melikechi. “Elemental analysis of laser induced breakdown spectroscopy aided by an empirical spectral database”, Applied Optics. 47, pp. G99-G104 (2008); creosa.desu.edu/LIBS.html).

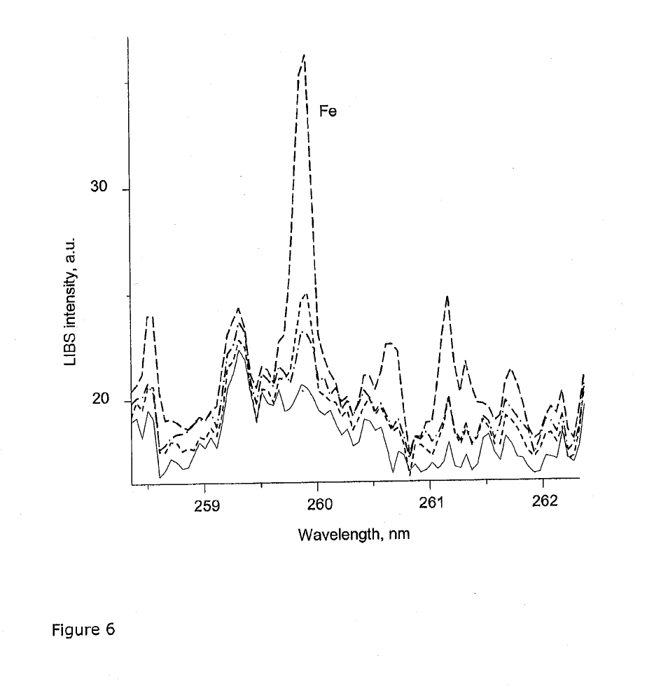

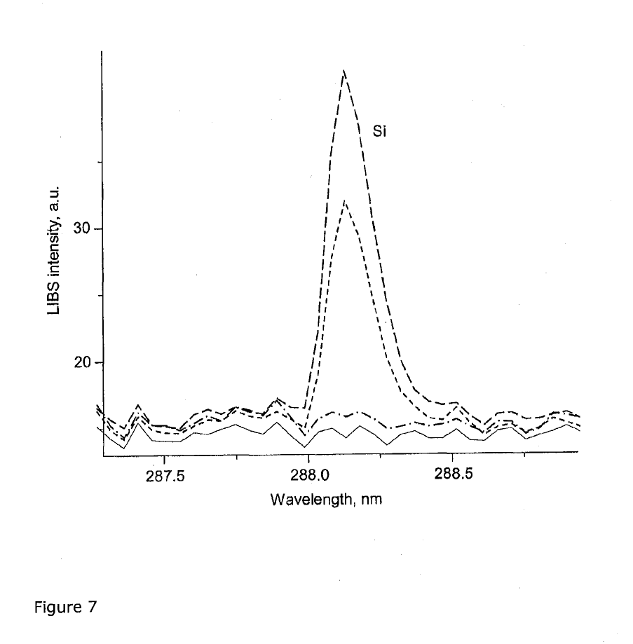

To prepare two-element-coded composite microparticles, 1.5 μm Fe-biotin particles suspended in phosphate buffered saline (PBS) were mixed with 3 μm diameter silicon particles modified by avidin (Si-avidin particles). After overnight incubation, unbound particles were removed by centrifugation through 5 μm pore filters. The filters containing the residue particles were examined by LIBS for the presence of Fe and Si elements (FIGS. 6 and 7, dashed line). The presence of both Fe (259.9 nm) and Si (288.1) related emission lines in the same sample demonstrated the presence of two-element-coded composite microparticles.

In control experiments, Fe-biotin particles were pre-incubated with an excess of avidin molecules. Following pre-incubation Fe-biotin particles and Si-avidin particles did not aggregate, demonstrating that nonspecific interactions between the two types of microparticles were negligible. Some silicon particles, having an average size of about 3 μm, were trapped by the 5 μm pore filters (FIGS. 6 and 7, dotted line).

In a second control experiment, iron oxide particles modified by biotin (Fe-biotin particles) were suspended in PBS and centrifuged through the 5 μm pore filters. This experiment tested for nonspecific binding of Fe-biotin particles to the test tube and the filter. Nonspecific binding was found to be insignificant (FIGS. 6 and 7, dotted-dashed line). Solid lines in FIGS. 6 and 7 represent LIBS spectra of empty filters.

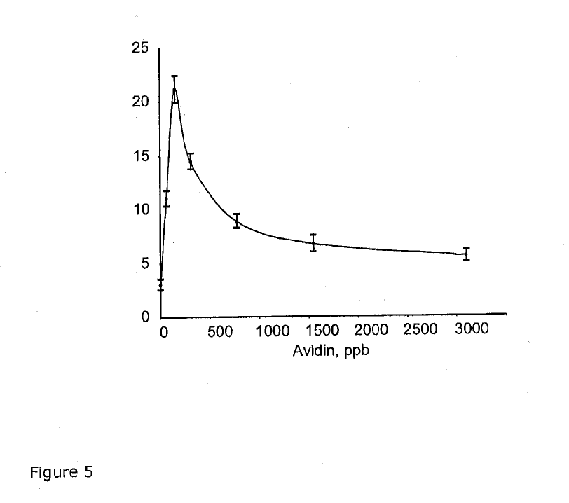

To estimate the sensitivity of the assay system, avidin molecules were detected and quantified by a LIBS-based one-element (iron oxide) microparticle assay (FIG. 5). Iron oxide microparticles (1.5 μm) coated with biotin were purchased from Bangs Laboratories. Particle aggregation was induced by the addition of avidin. The quantity of aggregates was monitored by taking 140 laser shots of the surface of the 5 μm pore filters following removal of the filtrate with unbound microparticles. FIG. 5 shows that avidin concentration is related to the intensity of the Fe emission line at 259.9 nm integrated over the filter surface. The LIBS iron oxide microparticle assay had a detection-limit of about 30 ppb of avidin.

### 6. LIBS Immunoassay for Detection of Leptin on a Base of Silicon

Leptin and IgG H86901M and IgG H86412M monoclonal antibodies to leptin. were purchased from BIODESIGN International (Saco, Me.). Monoclonal antibodies were biotinylated via an EZ-Link Sulfo-NHS-Biotinylation Kit (Pierce, Rockford, Ill.), prior to performing the immunoassay. Solutions were diluted with phosphate buffered saline (PBS) containing about 5% of bovine serum albumin (BSA).

Leptin was mixed with a combination of the IgG H86901M and IgG H86412M monoclonal antibodies. A suspension of 3 μm silicon particles modified with avidin, prepared as described in Example 5, was added to the premixed leptin/antibody solution and incubated for 3 h at room temperature. In a control experiment, a suspension of 3 μm silicon particles modified with avidin, prepared as described in Example 5, was added to the PBS solution containing about 5% of BSA and incubated for 3 h at room temperature. To separate single particles from aggregated particles, the resultant solutions were briefly vortexed then centrifuged in 0.5 mL test tubes equipped with 5 μm pore filters as described above. The filters containing the residual particles were checked by LIBS for the presence of Si elements. Aggregates were quantified as described in Example 5.

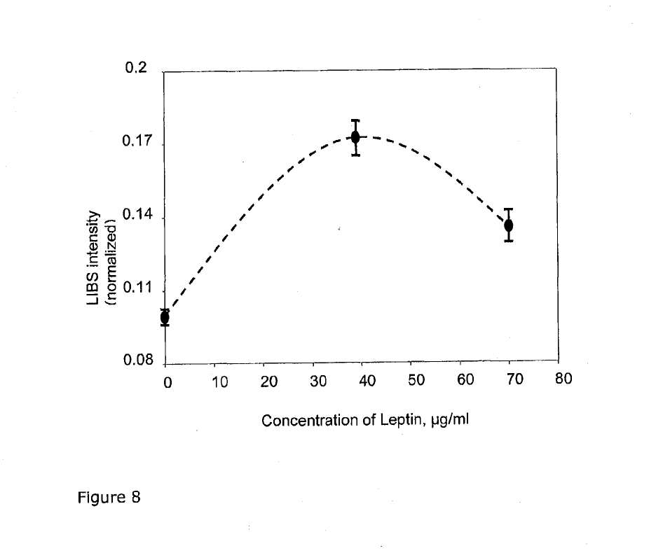

The intensity of the spectrum line for silicon at about 288.1 nm was normalized to the intensity of the spectrum line for carbon at about 247.8 nm (FIG. 8). FIG. 8 shows the leptin concentration represented by the normalized intensity of Si emission at about 288.1 nm, integrated over the filter surface. These results demonstrate the feasibility of mono- and multi-element-coded LIBS assays for the detection of proteins.

### 7. LIBS Immunoassay for Detection of CA 125 on a Base of Iron Oxide

CA 125 and IgG M86306M (Group A) and IgG M86429M (Group B) monoclonal antibodies to CA 125 were purchased from BIODESIGN International (Saco, Me.). Solutions were diluted as described above in Example 6.

One portion (about 100 μl) of iron oxide particles (1.5 μM) modified with protein G were added to the IgG M86306M (Group A) solution and another portion (about 100 μl) of iron oxide particles (1.5 μM) modified with protein G were added to the IgG M86429M (Group B) solution for overnight incubation at 4° C. Following incubation, unbound IgG molecules were washed away by three wash-centrifugation cycles using spin-filters with a pore size of about 100 nm (Millipore). CA 125 molecules of defined concentrations were added to a mixture of Fe particles from group A and Fe particles from group B in equal volumes and incubated overnight at 4° C. In a control experiment the PBS solution containing about 5% of BSA was added to a mixture of Fe particles from group A and Fe particles from group B in equal volumes and incubated overnight at 4° C.

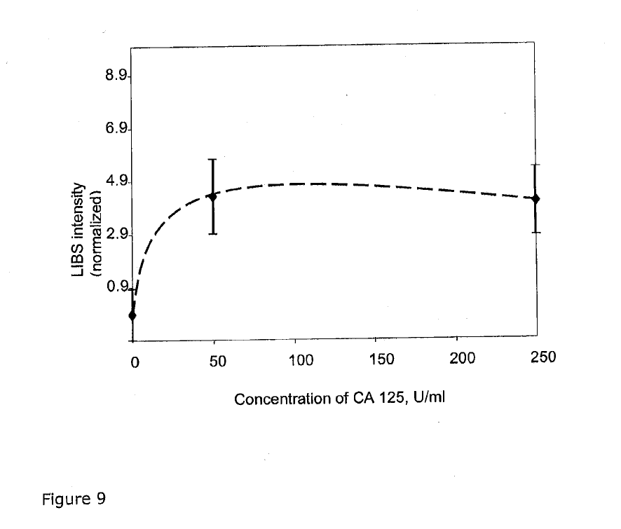

Single and aggregate particles were separated and residual particles on filters assayed as described in Example 6. The intensity of the iron spectrum at about 259.9 nm was normalized to the intensity of the carbon spectrum at about 247.8 nm, and the normalized LIBS intensity was plotted against the concentration of CA 125. FIG. 9 shows that mono and multi-element coded LIBS assays are feasible for detecting CA-125, a known marker for ovarian cancer, in a sample.

### 8. LIBS Immunoassay for Simultaneous Detection of Multiple Biomarkers

Multi- or mono-element coded particles are prepared and attached to specific antibodies as described above. Particles having the same element code are attached to a specific antibody for a particular biomarker. A mixture of element-coded particles bearing different codes and, accordingly, antibodies to different biomarkers, is prepared and added to a biological sample. The sample and element-coded particles are incubated to allow binding between each type of biomarker and its specific antibody. After incubation, unbound element-coded particles are removed from the sample as described in Example 4. Element-coded particles bound to the molecules of interest may also be removed. The sample is then analyzed by LIBS. Spectra are produced which identify and quantify each type of biomarker present in the sample.

### 9. Two-Element Coded (Si and Fe) Assay for Detection of CA 125

To perform immunoassay ovarian cancer biomarkers Leptin and CA 125 were used with pairs of monoclonal antibodies H86901M and H86412M for Leptin, M86306M and M86429M for CA 125 (Biodesign International). Monoclonal antibodies were biotinylated prior to doing assay. EZ-Link Sulfo-NHS-Biotinylation Kit (Pierce, Rockford, Ill.) was used for this purpose. All buffers used for dilutions contained about 5% of BSA to mimic blood conditions. To separate single and aggregated particles we used 0.5 mL test tubes equipped with 5 μm pore size filters (Millipore) or magnetizing. In the experiments with particle assays every step of incubation was followed by washing step to remove unbound reactants and then centrifuging step to separate single and aggregated particles. In control experiments the PBS buffer solution containing about 5% of BSA was added to a mixture of particles and incubated overnight at 4° C.

Iron oxide particles (1.5 μm) modified with protein G were added to the antibody M86306M (Group A) solution. Silicon particles (1 μm) modified with streptavidin were added to the antibody M86429M (Group B) solution for overnight incubation at 4° C. Following incubation, unbound antibody molecules were washed away by three wash-centrifugation cycles using spin-filters with a pore size about 100 nm (Millipore). CA 125 molecules of defined concentrations were added to a mixture of Iron oxide particles group A and Silicon particles group B in equal volumes and incubated overnight at 4° C.

Single and aggregated particles were separated using strong magnets (residual flux density about 14.5-14.8 KGs (K&J Magnetics, Inc. website, http://www.kjmagnetics.com/specs.asp. Accessed 7 Feb. 2011)) and residual particles were placed on filters.

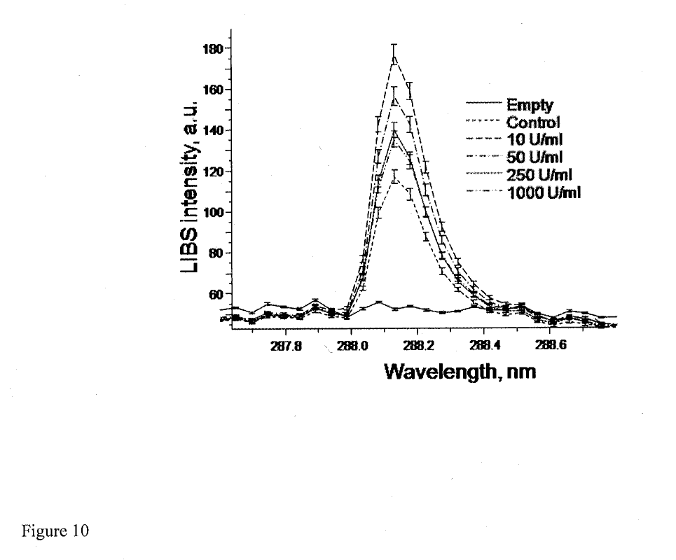

The magnetizing type of assay was employed. In this approach, following the incubation, the single silicon particles, the single iron oxide particles and particle aggregates were separated using strong magnets. After completing steps of magnetizing and pipetting, the residue particles left on the filters were analysed by LIBS for the presence of silicon. FIG. 10 shows the fragment of LIBS spectra around 288.1 nm silicon emission line obtained by the two-element (Si and Fe) Tag-LIBS assay for detection of CA 125 biomarker. The control lowest solid line on FIG. 10 was obtained from the empty filter. The dash line curve is a LIBS spectrum of control sample where instead of CA 125 the buffer was added. Other lines represent various concentrations of CA 125 in a solution (see FIG. 10).

### 10. Two-Element Coded (Au and Fe) Assay for Detection of Avidin in Human Blood Plasma

About 4 μg of 50 nm biotinylated Gold nano-particles (Nanocs, Inc.) and 50 μg of 1.5 μm Iron oxide particles modified with biotin (Bangs Lab) were added to about 0.75 ml human blood plasma (Blood Bank of Delmarva). Thawed human blood plasma has been filtered over 5 μm pore size filters for 1 min at relative centrifugal force 8,000×g. Not more than 0.25 mL PBS has been used to adjust volumes of samples. Avidin molecules of defined concentrations were added to the suspension of biotinylated Au nano-particles and biotinylated Iron oxide particles for overnight incubation at 4° C. Single and aggregated particles were separated by using strong magnets and residual particles on filters were assayed.

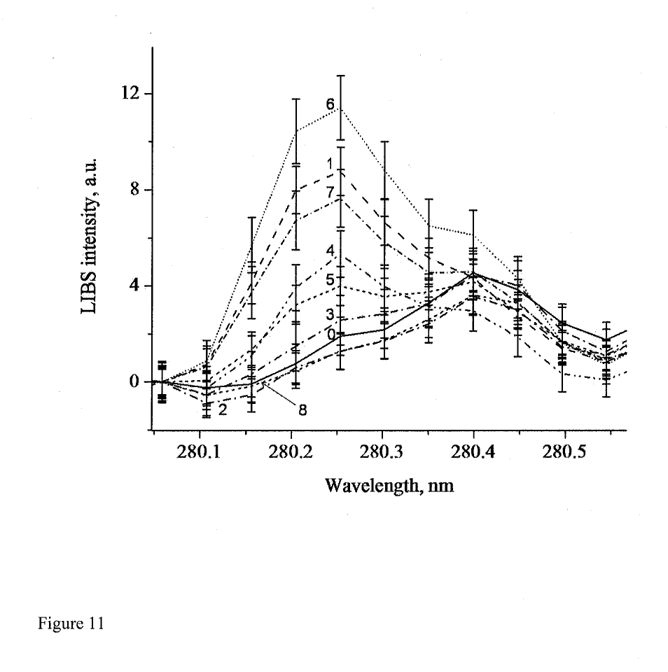

Result at FIG. 11 demonstrated the ability of Tag-LIBS approach to detect model molecules avidin in human blood plasma. Tag-LIBS analysis has been performed with a series of dilutions resulting in following final concentrations of avidin: 0 ppb, 6.4 ppb, 64.5 ppb, 322.4 ppb, 644.8 ppb, 1483.1 ppb, 2321.4 ppb, 3224.2 ppb, and 6448.4 ppb (curves 0-8, FIG. 11). The spectrum of the empty filter has been subtracted from the sample spectra. For purpose to simplify comparison of the Gold emission peak intensities at 280.2 nm the sample spectra have been slightly shifted along the Y axis (FIG. 11). Data of three Tag-LIBS experiments were averaged to plot the control curve (curve 0, FIG. 11). The lowest concentration of model protein avidin about 6 ppb with 8:1 signal-to noise ratio has been measured by Tag-LIBS approach in human blood plasma (curve 1, FIG. 11).

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A method of identifying a biomarker in a biological sample comprising the steps of a) reacting a biological sample containing a biomarker with a plurality of element-coded particles each comprising a compound that binds to the biomarker, b) removing unbound element-coded particles from the sample, and c) detecting the element-coded particles in the sample using an optical system.

2. The method of claim 1, further comprising step d) quantifying the element coded particles in the sample.

3. The method of claim 1, wherein the biological sample is a body fluid.

4. The method of claim 1, wherein the biological sample is selected from the group consisting of cells, tissues, culture media and organisms.

5. The method of claim 1, wherein the compound that binds to the biomarker is selected from the group consisting of proteins, oligonucleotides, polysaccharides, and lipids.

6. The method of claim 5, wherein the compound that binds to the biomarker is a protein selected from the group consisting of antibodies, antigens, receptors, ligands, biotinylated proteins, avidin-conjugated proteins and nucleic acid binding proteins.

7. The method of claim 1, wherein the optical system comprises a laser-induced breakdown spectrometer.

8. The method of claim 1, wherein the biomarker is a biomarker for a disease.

9. The method of claim 8, wherein the biomarker for a disease is a cancer biomarker.

10. The method of claim 1, wherein the average size of the element-coded particles ranges from 10 nm to 10 mm.

11. The method of claim 10, wherein the size of the element-coded particles ranges from 10 nm to 1 mm.

12. The method of claim 1, wherein the shape of the element-coded particle is selected from the group consisting of spheres, rods, tubes, rings, plates, bricks, strips, strings and threads.

13. The method of claim 1, further comprising the step of identifying and quantifying the unbound element-coded particles removed from the sample.

14. A method of identifying multiple biomarkers simultaneously in a biological sample comprising the steps of a) reacting a biological sample containing more than one biomarker with a plurality of element-coded particle types, wherein each particle type comprises a specific element code and a compound that binds to a discrete biomarker, c) removing unbound element-coded particles from the sample, and d) detecting the element-coded particles in the sample using an optical system.

15. The method of claim 14, further comprising step e) quantifying each type of element-coded particle in the sample.

16. The method of claim 14, wherein the optical system comprises a laser-induced breakdown spectrometer.

17. A method of tagging an object comprising the step of a) attaching one or more element-coded particle as a tag to the object to produce a tagged object.

18. The method of claim 17 further comprising steps b) analyzing the tagged object by laser-induced breakdown spectroscopy (LIBS) to produce an emission spectrum from the tag, and c) identifying the object by matching the tagged object with the emission spectrum of the tag.
